(19) United States
(12) Patent Application Publication
(10) Pub. No.: US 2022/0031911 A1
(43) Pub. Date: Feb. 3, 2022

(54) DEVICES AND METHODS FOR TREATING TISSUES, INCLUDING IRRADIATED TISSUES

(71) Applicant: LifeCell Corporation, Madison, NJ (US)

(72) Inventors: Christopher A. Campbell, Madison, NJ (US); Patrick S. Cottler, Madison, NJ (US)

(73) Assignee: LifeCell Corporation, Madison, NJ (US)

(21) Appl. No.: 17/298,886

(22) PCT Filed: Dec. 3, 2019
(86) PCT No.: PCT/US2019/064158

§ 371 Date: Jun. 1, 2021

Related U.S. Application Data
(60) Provisional application No. 62/774,477, filed on Dec. 3, 2018.

(51) Int. Cl. A61L 27/36

(52) U.S. Cl. CPC A61L 27/362; A61L 27/3641; A61L 2430/34; A61L 2430/04; A61L 27/3683

(57) ABSTRACT

The present disclosure provides devices and methods for treating a tissue site. The device and methods can be used to treat tissue that has been irradiated and can include a tissue matrix having the ability to support tissue regeneration and vascularization with cells from the tissue site.

This application claims priority under 35 U.S.C. § 119 to U.S. Provisional Application No. 62/774,477, which was filed on Dec. 3, 2018 and is incorporated by reference in its entirety.

The present disclosure relates to devices and methods for treating tissue with tissue matrices, including tissue that has been irradiated.

The use of acellular tissue matrices such as ALLODERM®, a dermal acellular matrix produced by LIFECELL® CORPORATION (Madison, N.J.), for use in breast procedures has become increasingly popular with plastic surgeons. Such materials provide a number of advantages and can be used to replace or augment supportive structures after, for example, mastectomy. Such materials can also be useful in reconstructive or aesthetic procedures (e.g., reconstruction or breast augmentation) by providing additional support for breast implants, allowing improved control of breast shape, preventing skin rippling, and/or preventing or treating other problems that may occur with breast augmentation (e.g., symmastia and bottoming out.)

Many patients who undergo surgical oncology procedures to treat breast or other tissues also receive radiation therapy. In some cases, radiation may damage healthy tissues, thereby slowing healing or impeding reconstructive procedures. The ability of tissue matrix materials to support tissue regeneration and vascularization in irradiated tissue, however, is not completely understood. Accordingly, there is a need for tissues matrices for breast or other irradiated tissues that have demonstrated the ability to support regeneration and/or revascularization even after irradiation.

The present application provides methods for treating irradiated tissues with tissue matrices that support vascularization and regeneration. The methods can include use of porcine dermal materials having the ability to support tissue growth, vascularization, and/or regeneration when used in conjunction with radiation therapy or after radiation.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 provides CD31 expression/high-powered field of view (HFV) for samples of porcine acellular dermal matrix from a mouse subcutaneous model implanted in irradiated and non-irradiated tissue as described in Example 1.

FIG. 2 provides smooth muscle actinin (SMA) expression/HFV for samples of porcine acellular dermal matrix from a mouse subcutaneous model implanted in irradiated and non-irradiated tissue as described in Example 1.

FIG. 3 provides vimentin expression/HFV for samples of porcine acellular dermal matrix from a mouse subcutaneous model implanted in irradiated and non-irradiated tissue as described in Example 1.

FIG. 4 provides CCR7 expression/HFV for samples of porcine acellular dermal matrix from a mouse subcutaneous model implanted in irradiated and non-irradiated tissue as described in Example 1.

FIG. 5 provides CD206 expression/HFV for samples of porcine acellular dermal matrix from a mouse subcutaneous model implanted in irradiated and non-irradiated tissue as described in Example 1.

FIG. 6 provides M2:M1 ratios for samples of porcine acellular dermal matrix from a mouse subcutaneous model implanted in irradiated and non-irradiated tissue as described in Example 1.

FIG. 7 provides photo acoustic microscopy (PAM) data demonstrating vessel coverage for samples implanted in irradiated and non-irradiated tissues at 3, 7, and 14 days.

FIG. 8 provides PAM data demonstrating oxygen saturation for samples implanted in irradiated and non-irradiated tissues at 3, 7, and 14 days.

FIG. 9 provides SEM images of a top surface of PADM used in the present study.

FIG. 10 provides SEM images of a side surface of PADM used in the present study.

### DETAILED DESCRIPTION

Reference will now be made in detail to various embodiments of the disclosed devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present disclosure relates generally to devices for surgical breast procedures and systems and methods relating to such devices. The devices and methods can be used for tissue augmentation, repair or regeneration of damaged tissue, and/or correction of tissue defects. As such, the devices, systems, and methods discussed herein can be suitable for a wide range of surgical applications, such as, for example, aesthetic surgery, breast reconstruction, breast augmentation, breast enhancement, breast reduction, and revisionary breast surgeries.

The tissue matrices used to produce the devices or perform methods described herein can include a variety of different materials. For example, an acellular tissue matrix or other tissue product can be selected to allow tissue ingrowth and remodeling to assist in regeneration of tissue normally found at the site where the matrix is implanted.

In one embodiment, the devices and methods include porcine acellular dermal matrices. Suitable porcine acellular dermal matrices can be produced by decellularization and further treatment to remove antigenic materials such as alpha-galactose moieties. In addition, in some embodiments, the PADM is treated to control tissue handling properties to more closely mimic elasticity and/or pliability of human tissue. An exemplary acellular dermal matrix that may be useful for the methods of the present disclosure is produced by LifeCell Corporation and is sold as Artia®.

The devices described herein can be used for treatment of a number of tissue sites, including tissue sites that have been subjected to clinical doses or radiation. For example, suitable tissue sites can include tissue in or around the breast, possibly in conjunction with surgical breast procedures such as mastectomy, lumpectomy, and/or reconstructive procedures following oncology procedures.

The methods disclosed herein can include selection of a tissue site in need of treatment, particularly a treatment site that has been exposed to a clinical dose of radiation. As discussed above, exemplary treatment sites can include tissue in or around a breast, including tissue that may have been subjected to a surgical procedure such as mastectomy (or variations thereof, such as radical mastectomy) or lumpectormy.

The devices and methods may include a reconstructive procedure and/or augmentation. For example, the methods can include closure of a surgical site, but may also include implantation of a tissue expander or breast implant. The methods will be understood to encompass use of the tissue matrices for pre-pectoral, subpectoral, or other known or developed reconstructive procedures.

It may also be desirable to provide tissue matrices that allow regeneration and vascularization without further modification to the tissue matrices. For example, it is sometimes considered desirable to produce fenestrations in tissue matrices prior to implantation. Accordingly, the present disclosure demonstrates the ability of PADM to provide for tissue ingrowth, regeneration, and/or vascularization without modification of the tissue to produce additional fenestrations or openings.

### Example 1

### mplantation of PADM in Mice With or Without Prior Irradiation Demonstrates Excellent Biointegration

A porcine acellular dermal matix (ARTIA, LIFECELL CORPORATION) (referred to for this example as PADM) was studied using a dorsal skinfold wound chamber model. Sixteen mice received an implantation of PADM. Prior to implantation, eight animals received a clinical dose of radiation (35 Gy) to the skin, and eight animals did not receive radiation (0 Gy). Implantation was performed twelve weeks after irradiation. Real-time evaluations of the vascular integration, hemoglobin, and oxygen saturation within the PADM were made and analyzed through repeated photoacoustic microscopy (PAM) over 14 days. The PAM method is described by DeGeorge et al., “Advanced Imaging Techniques for Investigation of Acellular Dermal Matrix Biointegration,” Plastic and Reconstructive Surgery, Vol. 139(2), 395-405 (February 2017). At the terminal time point, vascular ingrowth (CD31), fibroblast scar tissue formation (smooth muscle actin, vimentin) and inflammatory versus remodeling macrophage function (M2:M1 ratio) were evaluated by immunohistochemistry. SEM images of the PADM were also analyzed for levels of porosity.

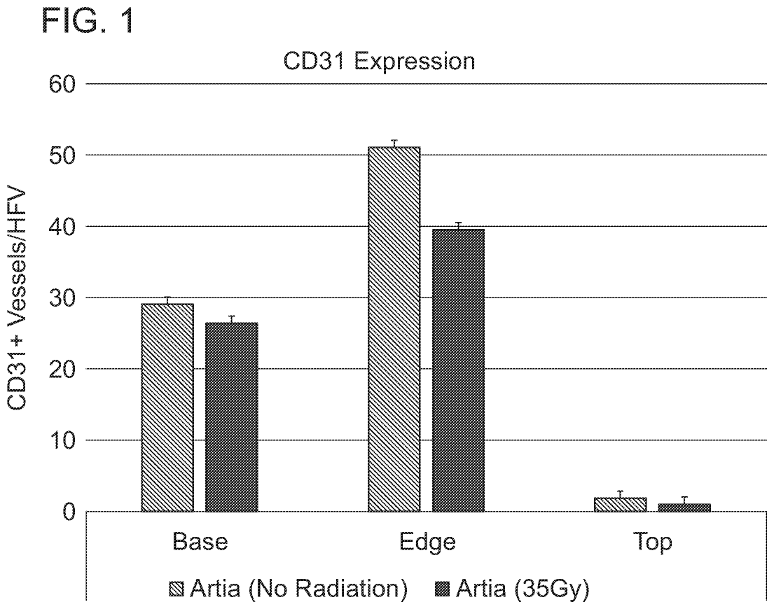

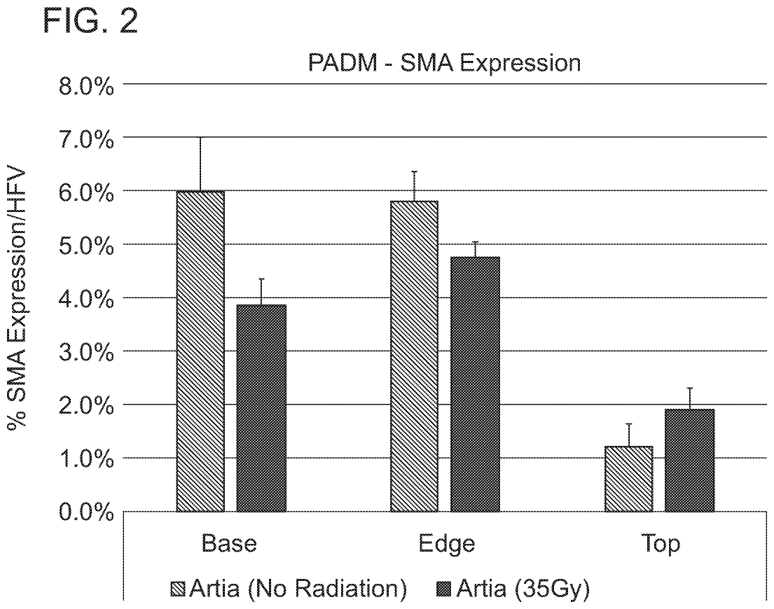

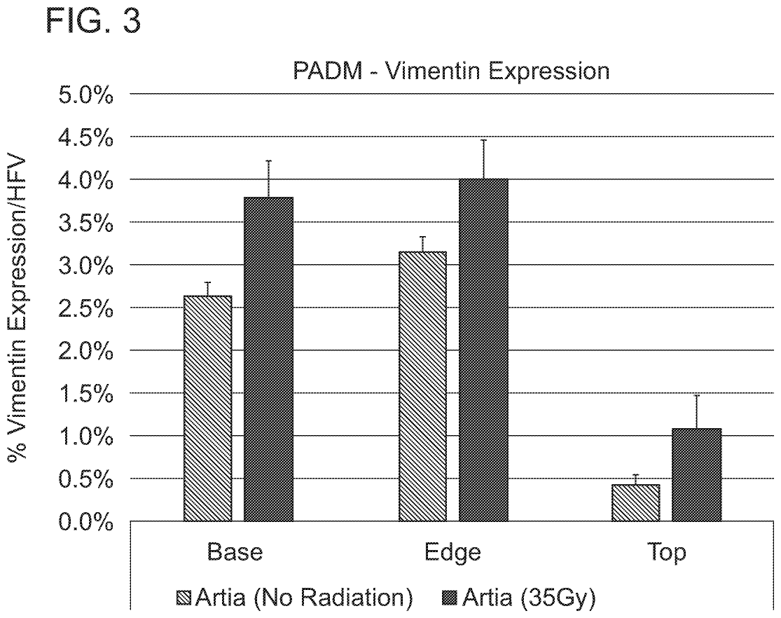

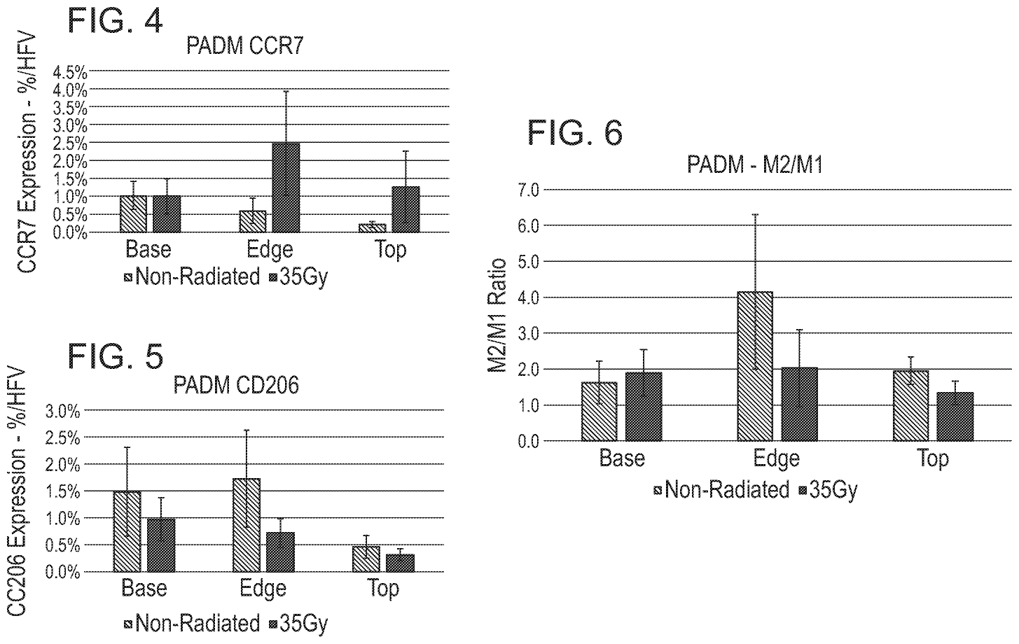

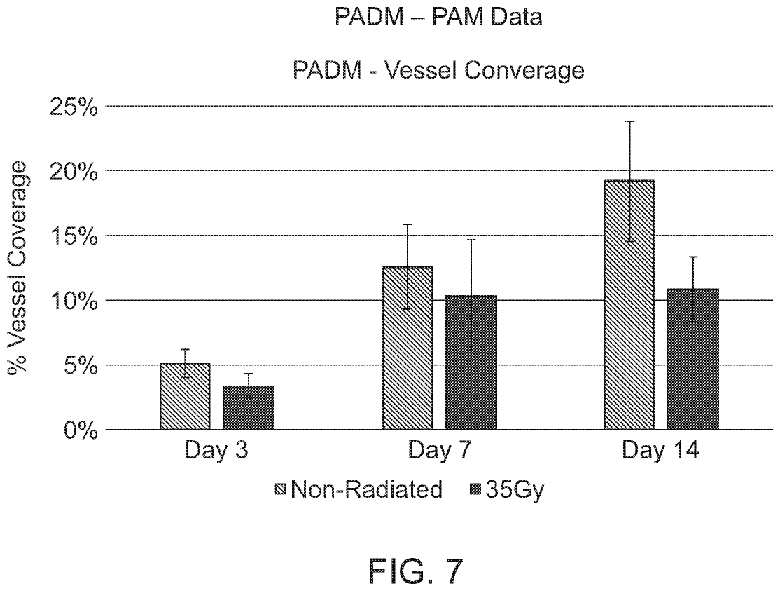

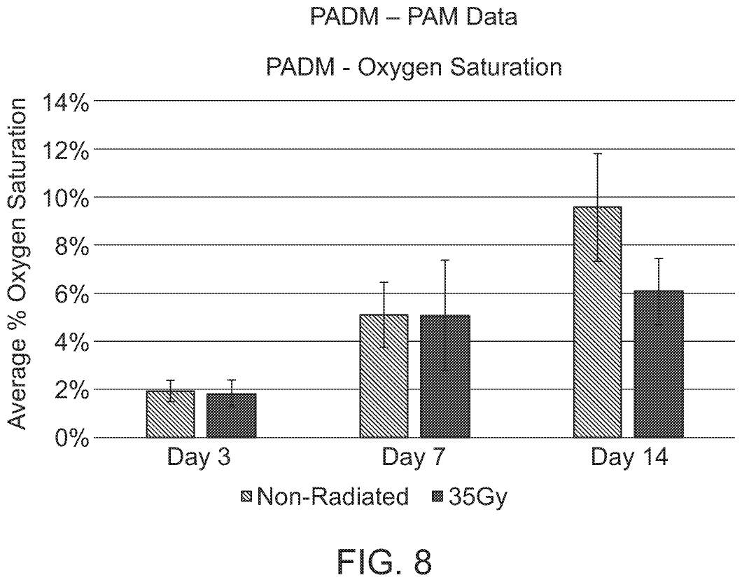

The PADM demonstrated vascularization and macrophage populations indicative of remodeling and regeneration. Using repeated PAM imaging through the window chamber, vascular ingrowth was shown to increase over 14 days, with a commensurate increase in hemoglobin and oxygen saturation within the ADM (FIGS. 7 and 8). Robust vessel integration as seen with CD31 staining (FIG. 1) with appropriately low SMA (FIG. 2) and vimentin (FIG. 3) expression consistent with low fibrosis. M2 machrophages were over-represented in the macrophage population consistent with a remodeling physiology (FIG. 4-6). Note that CCR7 is an accepted cell marker for M1 (inflammatory) macrophages and CD206 is an accepted cell marker of M2 (regenerative) macrophage. By calculating the % area in a high powered field of view that is positive for CCR7 (FIG. 4) and CD206 (FIG. 5) staining, the ratio of CD206 expression to CCR7 expression was determined (FIG. 6). Analysis of PADM performance in this model also demonstrates consistent performance in an irradiated skin envelope.

PADM used in the present study demonstrated increased hemoglobin and oxygen saturation content by 7-14 days consistent with the analysis of other collagen substrates. CD31 (FIG. 1) and SMA (FIG. 2) histological values demonstrate appropriately high vascularity and modest fibrosis. The PADM performed equally well in an irradiated field. Taken together the PADM product is considered well-suited for breast reconstruction.

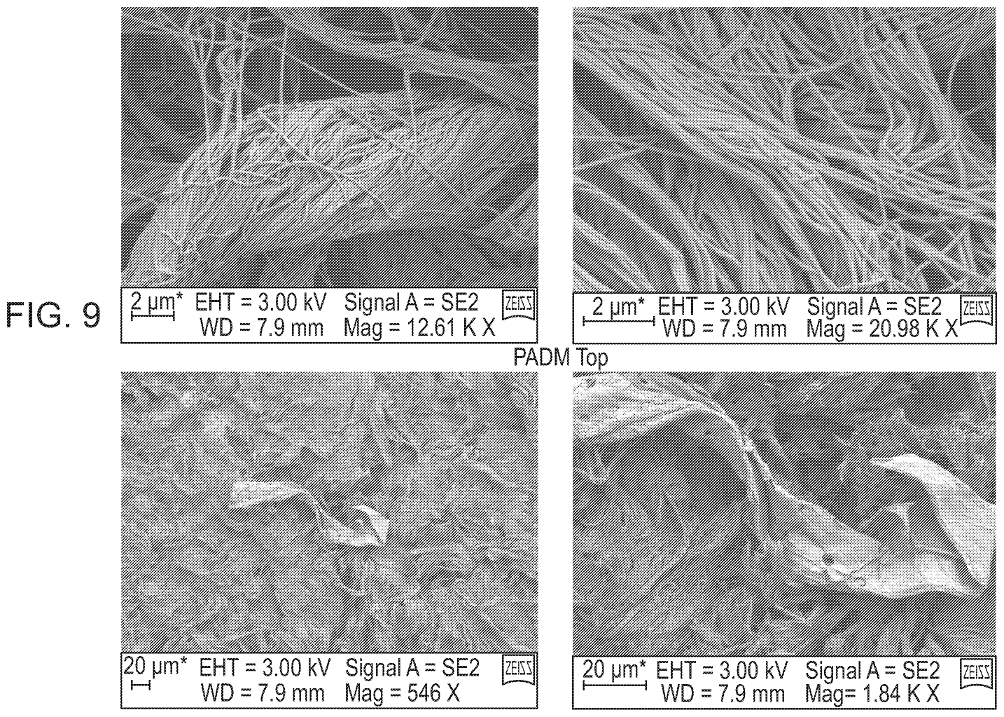

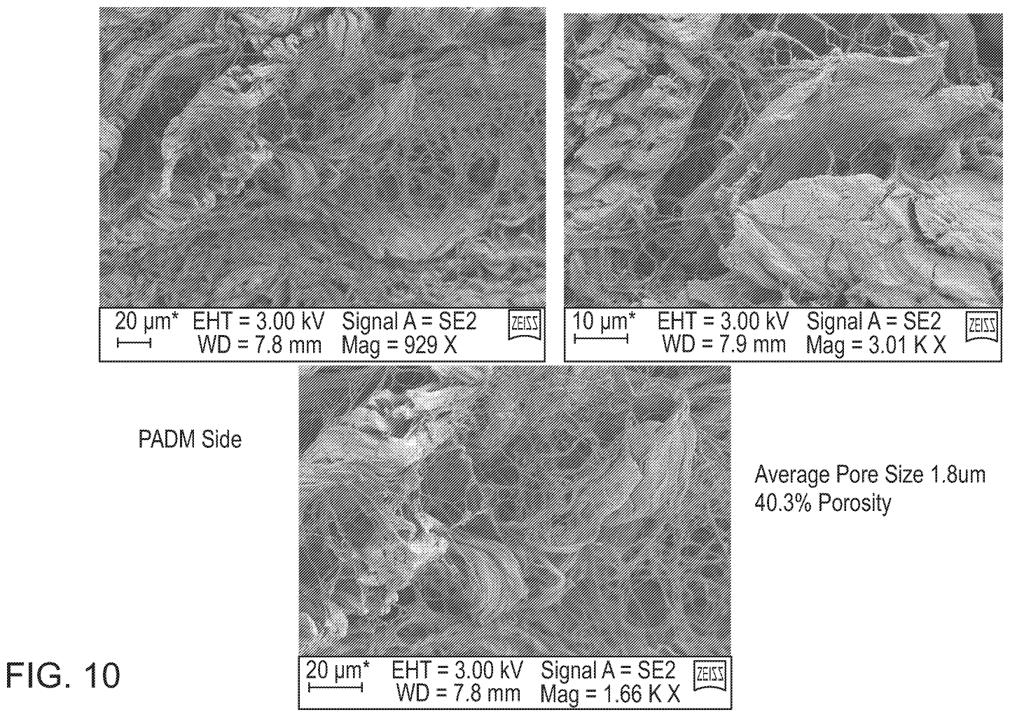

FIG. 9 provides SEM images of a top surface of PADM used in the present study. FIG. 10 provides SEM images of a side surface of PADM used in the present study. SEM demonstrates organized collagen fibrils with regularly positioned natural fenestrating pores with a 40.3% porosity to allow cellular ingrowth.

1. A method of treating a tissue site, comprising:
selecting a patient having a tissue site that has been irradiated; and
implanting in or near the tissue site a tissue matrix having the ability to support tissue regeneration and vascularization with cells from the tissue site.

2. The method of claim 1, wherein the tissue site is a breast.

3. The method of claim 2, wherein the tissue site is a breast that has undergone a surgical procedure.

4. The method of claim 3, wherein the surgical procedure includes a mastectomy or lumpectomy.

5. The method of claim 1, wherein implanting the tissue matrix includes implanting the tissue matrix around an implant or tissue expander.

6. The method of claim 1, wherein the tissue matrix is an acellular tissue matrix.

7. The method of claim 6, wherein the tissue matrix is a dermal tissue matrix.

8. The method of claim 6, wherein the tissue matrix is a porcine tissue matrix.

9. The method of claim 1, wherein the tissue matrix is in the form of a sheet.

10. The method of claim 1, wherein the tissue has been irradiated with external beam radiation.

11. The method of claim 1, wherein the tissue matrix is in the form of a sheet without mechanically formed fenestrations.
